(19) United States
(12) Patent Application Publication
(10) Pub. No.: US 2008/0207710 A1
(43) Pub. Date: Aug. 28, 2008

(54) Insulin Secretagogue Drugs

(75) Inventors: Juro Sakai, Tokyo (JP); Toshiya Tanaka, Tokyo (JP)

Correspondence Address: WENDEROTH, LIND & PONACK, L.L.P., 2033 K STREET N. W., SUITE 800, WASHINGTON, DC, 20006-1021, US

(73) Assignee: TOUDAI TLO, LTD., Tokyo (JP)

(21) Appl. No.: 11/630,112

(22) PCT Filed: Dec. 14, 2004
(86) PCT No.: PCT/JP04/18986

§ 371 Date: Dec. 20, 2006

(30) Foreign Application Priority Data
Jun. 23, 2004 (JP) 2004-184618

(51) Int. Cl. A61K 31/426

(52) U.S. Cl. 514/369

(57) ABSTRACT

Demanded is an insulin secretagogue that alleviates side effects, such as acceleration of obesity and cells so as not to be detrimental to insulin secretion β capability. The invention provides an insulin secretagogue agent not only dependent on glucose but also responsive to elevated blood sugar levels, comprising an effective amount of a substance having the activity of PPAR δ activation. This insulin secretagogue simultaneously exerts anti-obesity activity, serum cholesterol reducing activity useful for the treatment and prevention of arteriosclerosis, etc. and insulin resistance ameliorating activity.

### FIELD OF THE INVENTION

The present invention relates to hyperglycemia-responsive, glucose-dependent insulin secretagogue drugs which comprise an effective amount of at least one member selected from substances that activate or stimulate PPARδ.

### BACKGROUND OF THE INVENTION

Peroxisome proliferator activated receptor δ (also herein referred to as “PPARδ”) is a member of PPAR subtypes known as nuclear receptors involved in fatty acid metabolism. No tissue-specific expression pattern is observed for PPAR δ gene expression sites, and PPAR δ is near-ubiquitously expressed. PPAR δ is also designated as PPAR β or, for human, NUC-1, and lags behind other PPAR types, PPAR α and PPAR γ, in studying physiological functions. Only recently has the finding of a substance that activates or stimulates PPAR δ (hereinafter referred to as “PPAR δ agonist”) been obtained and disclosed for its pharmacological actions and pharmaceutical applications.

It has been disclosed that PPAR δ agonists are, for instance, 1) useful for raising plasma HDL levels to treat or prevent atherosclerotic cardiovascular diseases, and applicable in combination with one or more HMG-CoA reductase inhibitors to treat or prevent atherosclerotic cardiovascular diseases (see Patent Document 1: WO 97/28149, A), 2) useful as anti-diabetic therapeutics and anti-obesitic drugs (see Patent Document 2: WO 97/28115, A), 3) active in serum cholesterol lowering and LDL-cholesterol lowering (see Patent Document 3: WO 99/04815, A), and 4) active in elevation of HDL-cholesterol, and lowering of fibrinogen, triglycerides and insulin levels, and useful for preventing or treating dyslipidemia, syndrome X (including metabolic syndrome), heart failure, hypercholesteremia, cardiovascular disease, Type 2 diabetes mellitus, Type 1 diabetes mellitus, insulin resistance, hyperlipidemia, obesity, etc. (see Patent Document 4: WO 01/00603, A). Further, it has been disclosed that PPAR δ agonists enhance thermogenesis, uncoupling respiration in mitochondria and fatty acid β oxidation, etc., and useful as anti-diabetic agents, anti-obesitic agents, visceral accumulated fat-lowering agents and visceral fat accumulation-inhibiting agents (see Patent Document 5: WO 03/08967, A). In addition, it has been reported that GW501516 (compound name: 2-{2-methyl-4-[({4-methyl-2-[4-(trifluoromethyl)phenyl]-1,3-thiazol-5-yl}methyl)thio]-phenoxy}acetic acid; see Patent Document 4 and Non-Patent Document 1: Oliver et al., Proc. Natl. Acad. Sci., USA, 98, 5306-5311, 2001), known as a selective PPAR δ agonist, is observed to ameliorate obesity and insulin resistance in obese animals induced by high-fat dieting, and to improve diabetes as revealed by the decrease in plasma glucose and blood insulin levels in genetically obese animals (see Non-Patent Document 2: Tanaka et al., Proc. Natl. Acad. Sci., USA, vol. 100, 15924-15929, 2003).

The PPAR δ agonists are known to have the above-described pharmacological actions and pharmaceutical applications; with regard to insulin, however, only the action of ameliorating insulin resistance and of lowering insulin levels in non-hyperglycemic animals, disclosed in the aforementioned Patent Document 4 and Non-Patent Document 2, is discovered. Accordingly, no action thereof is known at all with respect to insulin secretion.

Insulin is a hormone secreted from pancreatic β cells depending on elevation of blood glucose levels, and known to have actions including increased protein synthesis in muscle and liver cells and stimulated glucose uptake and metabolism in adipose tissue.

Diabetes mellitus, a representative of insulin-related disorders, includes two forms of diabetes: diabetes mellitus type 1, characterized by deficiency in insulin secretion, and diabetes mellitus type 2, characterized by decreased sensitivity of body tissue to insulin. Anti-diabetic therapeutics applied at present include insulin preparations (e.g., animal insulin preparations extracted from bovine or porcine pancreas; human insulin preparations synthesized by genetic engineering techniques typically using Escherichia coli or yeasts, etc.), insulin sensitizers (e.g., troglitazone, pioglitazone, rosiglitazone, etc.), α-glucosidase inhibitors (e.g., voglibose, acarbose, miglitol, emiglitate, etc.), biguanides (e.g., phenformin, metformin, buformin, etc.), insulin secretagogues (sulfonylureas (e.g., tolbutamide, glibenclamide, gliclazide, chlorpropamide, tolazamide, acetohexamide, glyclopyramide, glimepiride, glipizide, glybuzole, etc.), repaglinide, senaglinide, nateglinide, mitiglinide, etc.), etc.

Among the aforementioned anti-diabetic therapeutics, the insulin sensitizer is applied to patients afflicted with persistent hyperglycemia, though blood insulin levels are elevated, to cause peripheral tissue to become more sensitive (more responsive) to insulin. In contrast, the insulin secretagogue stimulates pancreatic β cells to improve insulin secretion. Accordingly, both the drugs are clearly distinguishable each other in view of the site of action.
[Patent Document 1] WO 97/28149
[Patent Document 2] WO 97/28115
[Patent Document 3] WO 99/04815
[Patent Document 4] WO 01/00603
[Patent Document 5] WO 03/08967
[Non-Patent Document 1] Oliver et al., Proc. Natl. Acad. Sci., USA, 98, pp.5306-5311, 2001
[Non-Patent Document 2] Tanaka et al., Proc. Natl. Acad. Sci., USA, 100, pp.15924-15929, 2003

### SUMMARY OF THE INVENTION

As aforementioned, there are a variety of insulin secretagogues now used. The insulin secretagogues are considered to be most frequently prescribed anti-diabetic therapeutics. However, it has been found that they are accompanied with, in addition to hypoglycemia, severe side effects such as acceleration of obesity and arteriosclerosis. Accordingly, there is a great demand for developing insulin secretagogues (substances or agents that stimulate or induce insulin secretion) capable of relieving these side effects and reducing, as much as possible, a burden on pancreatic β cells so that the ability to secrete insulin may not be deteriorated.

The present inventors have conducted an extensive research on various compounds in order to solve the above problems. As a result, the present inventors have succeeded in finding that a substance that activates or stimulates PPAR δ acts directly on pancreatic β cells to stimulate potently glucose-dependent insulin secretion in response to hyperglycemia (or high blood sugar). The present inventors have continued the study to complete the present invention.

The present invention relates to the following:
[1] A hyperglycemia-responsive, glucose-dependent insulin secretagogue drug comprising an effective amount of at least one member selected from substances that activate or stimulate PPAR δ (PPAR δ activators or PPAR δ stimulants).
[2] The drug according to the above [1], wherein the PPAR δ-activating substance (PPAR δ activator) is selected from PPAR δ agonists.
[3] The drug according to the above [1] or [2], wherein the PPAR δ activator is 2-{2-methyl-4-[({4-methyl-2-[4-(trifluoromethyl)phenyl]-1,3-thiazol-5-yl}methyl)thio]phenoxy}acetic acid.
[4] A method for stimulating, not only responsively to hyperglycemia but also dependently on glucose, the secretion of insulin, which comprises administering an effective amount of a substance that activates or stimulates PPAR δ to a subject and stimulating hyperglycemia-responsive, glucose-dependent insulin secretion in said subject.
[5] A method for selecting a substance that stimulates the secretion of insulin not only responsively to hyperglycemia but also dependently on glucose, which comprises assaying a candidate substance for its ability to activate or stimulate PPAR δ, and selecting an identified PPAR δ-activating substance.

It is absolutely apparent that the insulin secretion-stimulating action (insulin secretagogue action) of the PPAR δ agonists, discovered herein by the present inventors, is pharmacologically different from the insulin sensitizing action (the action of ameliorating or decreasing resistance to insulin) thereof, already known in the prior art, and the site of insulin secretion-stimulating action is completely distinctive from that of insulin sensitizing action, as above-described. In addition, the already-known insulin level-lowering action of the PPAR δ agonists is the pharmacological action that is observed in non-hyperglycemic animals, as disclosed in Patent Document 4 and Non-Patent Document 2. Thus, it is extremely or near absolutely difficult for even a person skilled in the art to predict and infer the inventive PPAR δ agonist's insulin secretion-stimulating action (insulin secretagogue action) from these already known pharmacological actions.

### ADVANTAGEOUS PROFILES OF THE INVENTION

The present invention provides insulin secretagogues (compositions or agents) comprising an effective amount of substances that activate or stimulate PPAR δ, rather capable of not only ameliorating or reducing obesity and arteriosclerosis, which may occur as side effects induced by the prior art conventional insulin secretagogues, but also reducing a burden on pancreatic β cells as much as possible so that the ability to secrete insulin may not be deteriorated, and therapeutic methods comprising an application of said insulin secretagogue (composition or agent).

The present inventors have verified that, as revealed herein in Assay Examples below, the inventive effect, that is, the hyperglycemia-responsive, glucose-dependent insulin secretagogue action, is identified using extremely high-selective PPAR δ agonist, GW501516, against PPAR δ. It appears that GW501516 is a substance (compound) that has >1,000-fold selective affinity to PPAR δ even at very low concentrations on the order of nM over other nuclear receptors, PPAR α and PPAR γ (see Non-Patent Documents 1 and 2) and the inventive effect is attributable to its PPAR δ-activating action (PPAR δ-stimulating action).

The present invention allows the provision of insulin secretagogues capable of relieving side effects such as acceleration of obesity and arteriosclerosis and of minimizing, as much as possible, a burden on pancreatic β cells so that the ability to secrete insulin may not be deteriorated. The present invention also provides selection techniques for effective components serving as hyperglycemia-responsive, glucose-dependent insulin secretagogue drugs. According to the present invention, the hyperglycemia-responsive, glucose-dependent insulin secretagogue which comprises an effective amount of at least one member selected from substances that activate or stimulate PPAR δ can be applied to develop pharmaceutical drugs (or agents) that have anti-obesity action, not only serum cholesterol lowering action useful for treating or preventing arteriosclerosis but also insulin sensitizing action (insulin resistance-ameliorating or reducing action), thereby allowing the solution of said problems.

The above objects and other objects, features, advantages, and aspects of the present invention are readily apparent to those skilled in the art from the following disclosures. It should be understood, however, that the disclosures in the specification including the following best modes of carrying out the invention, examples, and others are illustrating preferred embodiments of the present invention and given for purposes of illustration only. It will become apparent to the skilled in the art that a great number of variations and/or alterations (or modifications) of this invention may be made based on knowledge from the disclosure in the following parts and other parts of the specification without departing from the spirit and scope thereof as disclosed herein. All of the patent publications and reference documents cited herein for illustrative purposes are hereby incorporated by reference into the present disclosure.

### BRIEF DESCRIPTION OF THE DRAWINGS

FIG. 1 shows assay results for activity of stimulating insulin secretion from pancreatic β cells when stimulated with glucose. GW: GW501516, DMSO: dimethyl sulfoxide, and G: glucose.

### BEST MODES OF CARRYING OUT THE INVENTION

The present invention provides the following:
1) A hyperglycemia-responsive, glucose-dependent insulin secretagogue drug comprising an effective amount of at least one member selected from substances that activate or stimulate PPAR δ, and
2) The drug according to the above 1), wherein the PPAR δ-activating or stimulating substance is 2-{2-methyl-4-[({4-methyl-2-[4-(trifluoromethyl)phenyl]-1,3-thiazol-5-yl}methyl)thio]phenoxy}acetic acid.

The aforementioned PPAR δ-activating or stimulating substance (PPAR δ activator or PPAR δ stimulant) is any as long as it is an agonist of PPAR δ. The preferable PPAR δ agonist is a substance (or compound) exerting, for example, in vitro activity of activating or stimulating PPAR δ at a dose of 3 mM or less.

Assays for the aforementioned PPAR δ-activating action (or PPAR δ-stimulating action), as can be used herein, include known methods and improved modifications thereof. Examples of such techniques used are the method using reporter systems wherein the DNA binding activity of yeast GAL4 protein is utilized (J. Biol. Chem., 270, pp.12953 to 12956, 1995), the method using reporter systems wherein the DNA binding domain of PPAR (peroxisome proliferator responsive element, PPRE) is utilized (Proc. Natl. Acad. Sci., USA, 91, pp.7355-7359, 1994; Proc. Natl. Acad. Sci., USA, 94, pp.4312-4317, 1997; J. Biol. Chem., 268(8), pp.5530-5534, 1993) and the method using reporter systems wherein the bacterial tetracycline operon is utilized (J. Biol. Chem., 270(41), pp.23975-23983, 1995) and others, as well as improved modifications thereof.

Preferred examples of the aforementioned PPAR δ-activating substances (or compounds) include known PPAR δ agonists. Representatives of said PPAR δ agonists include 2-{2-methyl-4-[({4-methyl-2-[4-(trifluoromethyl)phenyl]-1,3-thiazol-5-yl}methyl)thio]phenoxy}acetic acid (Glaxo, GW501516; WO 01/00603, J. Org. Chem., 63, pp.9116-9118, 2003), carbaprostacyclin (cPGI), L-165041 (WO 97/28115, J. Biol. Chem., 274, pp.6718-6788, 1999), YM-16638 (WO 99/04815), etc. Other representative PPAR δ-activating substances include those disclosed in the above-described Patent Documents 1 to 5 (WO 97/28149, WO 97/28115, WO 99/04815, WO 01/00603, WO 03/08967), other than the aforementioned preferred examples; substances disclosed in JP, A, 2001-354671 (benzoxazole derivatives, etc.); and substances disclosed in JP, A, 2003-171275 (pyrrole derivatives). Among them, preferable substances and compounds used herein are those exerting in vitro a PPAR δ-activating action (or PPAR δ-stimulating action) at doses of 3 mM or less.

Particularly preferable PPAR δ-activating substances suited for the present invention are those functioning as specific activators for PPAR δ. Among them, preferred substances include those substantially free of any activation and stimulation on PPAR α and PPAR γ out of PPAR members. Examples of such substances are those which, for example, at a dose of 1 μM or less, exert PPAR δ-activating or stimulating activity but are substantially free of any activation and stimulation on both of PPAR α and PPAR γ, in ligand assays using a chimera protein with the yeast transcription factor, GAL4.

The aforementioned hyperglycemia-responsive, glucose-dependent insulin secretagogue refers to drugs or agents that stimulate or induce insulin secretion not only in response to hyperglycemic conditions induced by a diet, etc., but also in proportion to the degree of blood glucose level elevation. The assay example disclosed herein below has clearly revealed that the insulin secretagogue drugs of the present invention stimulate insulin secretion from the pancreatic β cell in response to high glucose stimulation.

The present invention provides not only hyperglycemia-responsive, glucose-dependent insulin secretagogues (insulin secretagogue agents or compositions) which comprise an effective amount of at least one member selected from said substances that have the action of activating or stimulating PPAR δ but also methods for selecting a substance that stimulates or induces insulin secretion not only responsively to hyperglycemia but also dependently on glucose levels, which comprise assaying a candidate substance for its activity of activating or stimulating PPAR δ and selecting an identified PPAR δ-activating or stimulating substance. Such selection techniques are extraordinarily useful in the screening and selection of substances that stimulate or induce insulin secretion.

The insulin secretagogues of the present invention may be applied to patients afflicted with hyperglycemic conditions due to any of diabetes mellitus, metabolic syndrome, Cushing's syndrome, thiazide-induced glucose intolerance, malignant hypertension, burns, wounds, gigantism (giantism), angina pectoris, hyperthyroidism, bone fractures, surgery, emotional stress, myocardial infarction, metabolic diseases, central nervous system (CNS) diseases, endocrine diseases, pregnancy, brain tumors, subarachinoid hemorrhage, adrenal medullary tumors, acromegaly, pancreatic diseases and others, or with hypofunction of insulin secretion due to pituitary insufficiency and adrenal deficiency where said patients require insulin secretagogues.

Dose levels of the insulin secretagogues according to the present invention will vary depending on subjects to be treated, administration routes, target disorders, body weight, conditions of diseases and others. For instance, when the drug is administered orally to an adult diabetes mellitus patient requiring an insulin secretagogue, a single dose of effective components, PPAR δ activators or PPAR δ stimulants, usually ranges from about 0.01 to 800 mg, preferably 0.1 to 500 mg, and more preferably 0.5 to 300 mg. It is preferable that this dosage is administered 1 to 3 times a day.

The insulin secretagogue drugs of the present invention are administered orally, via oral cavity, by inhalation, through pernasal, permucosal, and per rectum routes and by injection. The active components, PPAR δ activators and PPAR δ stimulants, can be formulated in admixture with one or more suitable pharmaceutical additives. Embodiments of such known pharmaceutical additives may be suitably selected from components, conventionally used according to administration routes and applications of pharmaceutically formulated forms, as disclosed in, for example, “14th Edition Nippon Yakkyokuho (The Japanese Pharmacopoeia 14th Edition”, Japan (hereinafter referred to as “JPXIV”) and “Iyakuhin Tenkabutsu Jiten” (Pharmaceutical Excipient Dictionary), 1^{st} Edition, The Yakuji Nippo Limited (Jan. 14, 1994). The pharmaceutical additives may be selected from carriers, adjuvants, vehicles, excipients, binders, disintegrants, lubricants or glidants, coatings, humectants, wetting agents, solvents, bases, dispersers, emulsifiers, solution adjuvants, preservatives, stabilizers, and other materials, as disclosed in the above documents, suited for tablets, granules, powders, hard capsules, soft capsules, troches, dry syrups, syrups, solutions, suspensions, injections, aerosols, suppositories, etc. Specific examples of said pharmaceutical additives are starch, corn starch, crystalline cellulose, lactose, sucrose, glucose, mannitol, sorbitol, gelatin, agar, gum arabic, hydroxy-propylcellulose (HPC), low-substituted hydroxypropylcellulose (L-HPC), methylcellulose, carboxymethylcellulose, polyvinylpyrrolidone, carboxymethylcellulose sodium, carboxymethylcellulose calcium, magnesium stearate, talc, calcium carbonate, sodium hydrogen carbonate, hydrogenated vegetable oils, Macrogol, glycerin, water for injection, alginic acid, Polysorbate, lecithin, etc.

The insulin secretagogue drugs or agents of the present invention can be made according to any of methods for preparation of tablets, granules, powders, hard capsules, soft capsules, troches, dry syrups, syrups, solutions, suspensions, injections, aerosols, and suppositories, as disclosed in JPXIV.

The insulin secretagogue drugs of the present invention comprise an effective amount of at least one member selected from substances that activate or stimulate PPAR δ. Since it is revealed that PPAR δ agonists have anti-obesity activity as already pointed out, serum cholesterol lowering activity (useful for the therapy and prophylaxis of arteriosclerosis), LDL-cholesterol lowering activity, HDL-cholesterol elevating activity, etc., the inventive drugs and agents can be applied at quite low risk of inducing obesity and arteriosclerosis (severe side-effects derived by the prior art insulin secretagogues), and serve as extremely useful and advantageously effective insulin secretagogues for patients. In addition, the PPAR δ agonists have already been recognized to have activity of ameliorating insulin resistance in peripheral fat cells and skeletal muscles as aforementioned. This indicates that insulin will be secreted by stimulation with the PPAR δ agonist from pancreatic β cells in response to elevated blood glucose levels, efficiently spent in fat cells, and secreted without a burden on the pancreatic β cell. Therefore, the inventive insulin secretagogue drugs comprising an effective amount of at least one member selected from compounds that activate or stimulate PPAR δ can reduce risk of side effects, such as obesity and arteriosclerosis, observable in association with the prior art insulin secretagogues, and also act as insulin secretagogues not only capable of protecting the ability to secrete insulin from pancreatic β cells but also very effective for patients.

### EXAMPLES, ETC.

Details of the present invention are described by the following examples but such examples are provided only for illustrative purposes, and for referential embodiments of the present invention. These examples have been described herein for the purpose of illustrating specific embodiments of the present invention but should not be construed as in any sense limiting the scope of the invention disclosed herein. It should be understood in the present invention that various embodiments can be made or executed within the spirit, scope and concept disclosed herein. All the examples were carried out or can be carried out, unless otherwise disclosed herein specifically, by standard techniques which are well known and conventional to those skilled in the art. Unless otherwise specified, specific operations and treating conditions used in the following assay example and formulation example are done, when commercially available reagents and kits are utilized, based on protocols and chemicals attached to said kits.

### Assay Example

### Glucose-Stimulated Insulin Secretion

### 1) Materials and Methods

A test compound used herein, GW501516, that is, 2-{2-methyl-4-[({4-methyl-2-[4-(trifluoromethyl)phenyl]-1,3-thiazol-5-yl}methyl)thio]phenoxy}acetic acid, was synthesized according to Example 65 and 66 steps of WO 01/00603, A.

Animals, 6-week-old male C57BL/6J and Lepr^{db}/Lepr^{db} mice, were purchased from Clea Japan, Inc., and kept under the following conditions: room temperature, 25° C.; humidity, 60±10%; and 12 hr light/dark cycles (light period, 08:00 to 20:00). All animals were tamed for one week and then subjected to tests. The animals were given free access to food (commercial lab chow, pellet form CE-2, Clea Japan, Inc.) and water.

Male C57BL/6J and Lepr^{db}/Lepr^{db} mice were used. After clamping the common bile duct at a point close to the duodenum outlet under Nembutal anesthetization, 1 mg/mL collagenase (Wako Pure Chemical Industries, Ltd., Japan) was injected into the common bile duct at a liver side. The pancreas was taken out and incubated at 37° C. for 13 min. Thereafter, Hank's balanced salt solution was added, the pancreas tissue suspension was shaken and then centrifuged to remove supernatants. The pancreas tissue was resuspended in Hank's balanced salt solution, and then subjected to filtration followed by addition of Hank's balanced salt solution to make the tissue suspension volume 35 mL. The resultant tissue suspension was mixed and then allowed to stand for 5 min. Supernatants were removed with an aspirator except that 5 mL of the solution was retained, and the remaining solution was remixed with Hank's balanced salt solution to make the tissue suspension volume 35 mL. After this operation was repeated 12 times, the islets of Langerhans were collected under a microscope, and incubated in RPMI1640 (GIBCO) containing DMSO (0.1%), 10 or 100 nM GW501516 (final conc.)—added 10% fetal bovine serum (FBS, Sigma), 100 units/mL penicillin G sodium and 100 μg/mL streptomycin sulfate (GIBCO) for 24 hr. After incubation in a Krebs-Ringer bicarbonate buffer (KRBB, 119 mM NaCl, 4.74 mM KCl, 2.54 mM CaCl₂, 1.19 mM MgCl₂, 1.19 mM KH₂PO₄, 25 mM NaHCO₃, and 10 mM HEPES, pH 7.4) containing 2.8 mM glucose at 37° C. for 1 hr, the islets of Langerhans were dispensed in a 24-well plate at 10 islets per well, and incubated in KRBB containing 2.8, 5.6 or 16.7 mM glucose, respectively, at 37° C. for 1 hr. Each supernatant sample collected was assayed for insulin with rat insulin [¹²⁵I] Biotrak Assay System (Amersham).

### 2) Results

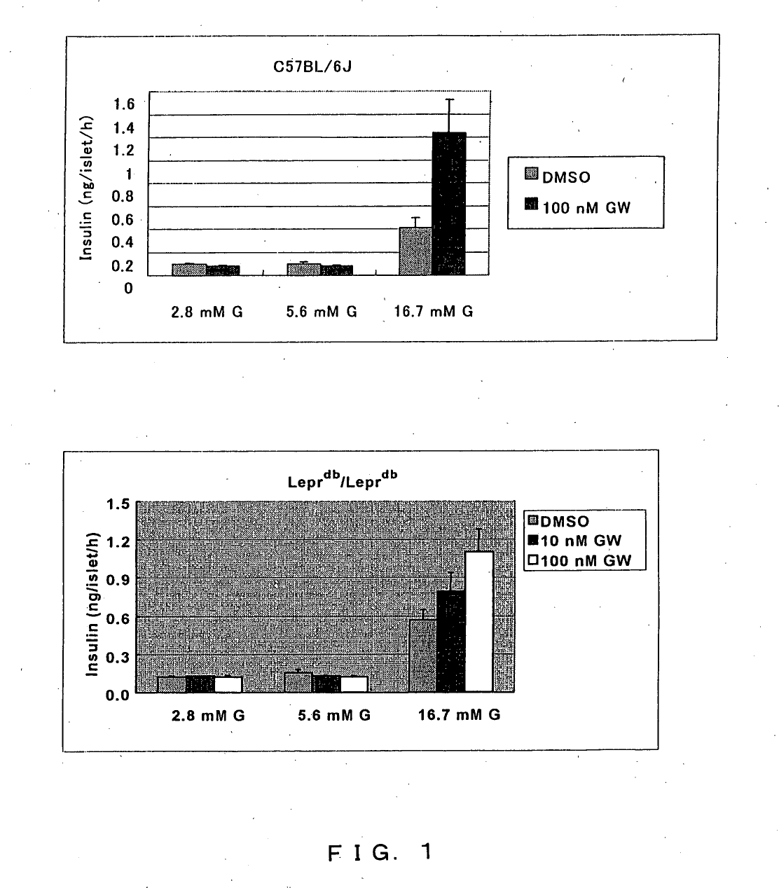

As shown in FIG. 1, among GW501516-treated groups, both C57BL/6J and Lepr^{db}/Lepr^{db} mice were observed to reveal that stimulation with 16.7 mM glucose resulted in significant insulin secretion. GW501516 increased dose-dependently insulin secretion from pancreatic δ cells when stimulated with elevated glucose levels.

### Example 1

A formula for one tablet (total amount per tablet 100 mg) is given below: 2-{2-methyl-4-[({4-methyl-2-[4-(trifluoromethyl)phenyl]-1,3-thiazol-5-yl}methyl)thio]phenoxy}acetic acid 5 mg, crystalline cellulose 70 mg, cornstarch 23 mg, and magnesium stearate 2 mg

The ingredients were formulated into tablets by known methods according to general pharmaceutical rules prescribed in JPXIV.

### INDUSTRIAL APPLICABILITY

The present invention provides techniques utilizable for development of pharmaceutical agents (drugs) and therapeutic methods wherein the PPAR δ activators or PPAR δ stimulants are used to stimulate or induce insulin secretion not only in response to hyperglycemia (high levels of sugar in the blood) but also dependently on glucose levels. The present invention allows insulin secretagogues capable of reducing adverse effects such as induction or acceleration of obesity and arteriosclerosis, and of minimizing, as much as possible, a burden on pancreatic β cells so that the ability to secrete insulin may not be deteriorated. Said hyperglycemia (high blood sugar)-responsive, glucose-dependent insulin secretagogue comprising an effective amount of at least one member selected from substances that activate or stimulate PPAR δ is expected to promise anti-obesity action, serum cholesterol lowering action and others useful for therapy and prophylaxis of arteriosclerosis, and insulin resistance reducing action, whereby it will be considered available for expanding and developing diabetes control and therapeutic techniques.

While the present invention has been described specifically in detail with reference to certain embodiments and examples thereof, it would be apparent that it is possible to practice it in other forms. In light of the disclosure, it will be understood that various modifications and variations are within the spirit and scope of the appended claims.

What is claimed is:

1. A hyperglycemia-responsive, glucose-dependent insulin secretagogue drug comprising an effective amount of at least one member selected from substances that activate PPAR δ.

2. The drug according to claim 1, wherein the PPAR δ-activating substance is 2-{2-methyl-4-[({4-methyl-2-[4-(trifluoromethyl)phenyl]-1,3-thiazol-5-yl}methyl)thio]phenoxy}acetic acid.

3. A method for selecting a substance that stimulates the secretion of insulin not only responsively to hyperglycemia but also dependently on glucose, which comprises assaying a candidate substance for its ability to activate PPAR δ, and selecting an identified PPARδ-activating substance.
